(19) United States
(12) Patent Application Publication
(10) Pub. No.: US 2026/0027297 A1
(43) Pub. Date: Jan. 29, 2026

(54) DRUG INFUSION DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/997,801

(22) PCT Filed: Aug. 28, 2023
(86) PCT No.: PCT/CN2023/115289

§ 371 Date: Jan. 23, 2025

(30) Foreign Application Priority Data
Aug. 3, 2022 (WO) PCT/CN2022/110057

(51) Int. Cl. A61M 5/172; A61M 5/142; A61M 5/145

(52) U.S. Cl. CPC A61M 5/172; A61M 5/14236; A61M 5/14248; A61M 5/1454; A61M 2005/14506; A61M 2205/0216; A61M 2205/0233; A61M 2205/18; A61M 2205/8206

(57) ABSTRACT

A drug infusion device includes a control mechanism, provided with first electrical contacts exposed to a surface of the control mechanism; and an infusion mechanism, including a housing, and the infusion mechanism being provided with second electrical contacts exposed on a surface of the housing. When the control mechanism and the infusion mechanism are assembled with each other, the first electrical contacts and the second electrical contacts press against each other, thereby electrically connecting the control mechanism with the infusion mechanism. An end of the first electrical contact is flush with the control mechanism surface or protruding from the surface of the control mechanism in contact with the first electrical contact. When the control mechanism and the infusion mechanism are assembled, the first electrical contact and the second electrical contact may be contacted preferentially, the reliability of the electrical connection between the control mechanism and the infusion mechanism is improved.

### CROSS-REFERENCE TO RELATED APPLICATION

This application claims the priority benefit of PCT application no. PCT/CN2022/110057, filed on Aug. 3, 2022. The entirety of the above-mentioned patent application is hereby incorporated by reference herein and made a part of this specification.

### TECHNICAL FIELD

The present invention mainly relates to the field of medical instruments, in particular to a drug infusion device.

### BACKGROUND

A drug infusion device is a medical device that achieves the purpose of disease treatment by continuously infusing drugs into the patient's body. Drug infusion devices are widely used in the treatment of diabetes. Insulin is continuously infused into the patient's skin according to the dosage required by the human body, to simulate the secretory function of the pancreas, thereby maintaining the stability of the patient's blood glucose. The drug fluid is usually stored inside the infusion device. In the existing drug infusion device, the infusion device is usually pasted directly on the patient's body through a medical tape, and the patient operates a remote device for infusion.

At present, the reliability of the electrical connection between the control mechanism module and the infusion mechanism module of the present drug infusion device is poor, which affects the normal use of the infusion device.

Therefore, there is an urgent need in the prior art for a drug infusion device that improves the reliability of the electrical connection between the control mechanism module and the infusion mechanism module.

### BRIEF SUMMARY OF THE INVENTION

The invention discloses a drug infusion device, the control mechanism module and the infusion mechanism module are electrically connected to each other by a plurality of first electrical contact and a plurality of second electrical contact that are press againsted each other, optimizing the circuit design and improving the reliability of the electrical connection. In particular, an end portion of the first electrical contact protruds from the surface of the control mechanism module in contact with the first electrical contact, so that when the control mechanism module and the infusion mechanism module are assembled, the first electrical contact and the second electrical contact may be contacted preferentially, further improving the reliability of the electrical connection between the control mechanism module and the infusion mechanism module.

The invention discloses a drug infusion device includes a control mechanism module, provided with a plurality of first electrical contacts exposed to a surface of the control mechanism module; and an infusion mechanism module, comprising a housing, and the infusion mechanism module being provided with a plurality of second electrical contacts exposed on a surface of the housing, when the control mechanism module and the infusion mechanism module are assembled with each other, the first electrical contacts and corresponding the second electrical contacts press againsted each other, thereby electrically connecting the control mechanism module with the infusion mechanism module; an end of the first electrical contact is flush with the control mechanism module surface or protruding from the surface of the control mechanism module in contact with the first electrical contact.

According to one aspect of the present invention, the control mechanism module is provided with a concave, the concave is provided with a recess, and the end of the first electrical contact protrudes from a surface of the recess.

According to one aspect of the present invention, the control mechanism module is provided with a concave, and the end of the first electrical contact protrudes from a surface of the concave.

According to one aspect of the present invention, the first electrical contact or the second electrical contact is a rigid metal contact or an elastic conductive member.

According to one aspect of the present invention, the elastic member is is a conductive spring, a conductive leaf spring, a conductive rubber or a conductive silicone.

According to one aspect of the present invention, t the first electrical contact is a rigid metal contact, the second electrical contact is an electrically conductive spring, an end of the rigid metal contact is provided with an arcuate end, and when the control mechanism module and the infusion mechanism module are assembled with each other, the end of the rigid metal contact is inserted into an inner cavity of the electrically conductive spring.

According to one aspect of the present invention, the housing is provided with a convex portion for assembly with the concave, and the convex portion is provided with a second groove to avoid interference when the end of the first electrical contact protrudes from the surface of the concave.

According to one aspect of the present invention, aa non-enclosed buzzer is provided in the control mechanism module.

According to one aspect of the present invention, a sound-permeable outlet is provided in the housing of the control mechanism module, and a waterproof sound-permeable membrane is disposed between the sound-permeable outlet and the buzzer.

According to one aspect of the present invention, the infusion mechanism module and the control mechanism module are detachable, and the control mechanism module is reused.

According to one aspect of the present invention, the infusion mechanism module and the control mechanism module are disposed of in one housing, discarded together after a single-use.

According to one aspect of the present invention, it further comprises an adhesive patch, the adhesive patch comprises a tape and a protective film, dense ventilation holes are provided on a surface of the tape and arranged evenly.

According to one aspect of the present invention, the infusion mechanism module further comprises a driving unit, a driving wheel and a reservoir, the reservoir is provided with a screw and a piston, a movement of the driving unit drives the driving wheel to rotate, which in turn drives the screw and the piston to move.

According to one aspect of the present invention, the infusion mechanism module further comprises a circuit board or a three-dimensional circuit coated on part of a surface of a structure, and the circuit board comprises a flexible circuit board or a rigid circuit board.

According to one aspect of the present invention, further comprises a power supply, and the power supply is a double-row button battery.

According to one aspect of the present invention, the infusion mechanism module further comprises an elastic conductor, the elastic conductor is electrically connected to the power supply and a specific connection end on the circuit board or three-dimensional circuit.

According to one aspect of the present invention, the infusion mechanism module further comprises a conductive tower-spring, one end of the conductive tower-spring is abutted to the driving unit, an other end of the conductive tower-spring is connected to the specific connection ends on the circuit board or three-dimensional circuit.

Compared with the prior art, the technical solution of the present invention has the following advantages:

In the drug infusion device, the control mechanism module is provided with a plurality of first electrical contacts exposed to a surface of the control mechanism module, the infusion mechanism module is provided with a plurality of second electrical contact. Firstly, the small contact area of the electrical contacts allows for flexible design and effective reduction of the size of the control mechanism module. Secondly, the control mechanism module and the infusion mechanism module are electrically connected to each other by a plurality of first electrical contact and a plurality of second electrical contact that are press againsted each other, optimizing the circuit design and improving the reliability of the electrical connection. Finally, the end portion of the first electrical contact protruds from the surface of the control mechanism module in contact with the first electrical contact, so that when the control mechanism module and the infusion mechanism module are assembled, the first electrical contact and the second electrical contact may be contacted preferentially, further improving the reliability of the electrical connection between the control mechanism module and the infusion mechanism module.

Furthermore, the elastic member act as the electrical contact is a conductive spring, a conductive leaf spring, a conductive rubber or a conductive silicone, conductive members with elasticity can further increase the reliability of the electrical connection.

Furthermore, the first electrical contact is a rigid metal contact, the second electrical contact is an electrically conductive spring, an end of the rigid metal contact is provided with an arcuate end, and when the control mechanism module and the infusion mechanism module are assembled with each other, the end of the rigid metal contact is inserted into an inner cavity of the electrically conductive spring, further improving the reliability of the electrical connection between the control mechanism module and the infusion mechanism module.

Furthermore, a non-enclosed buzzer is provided in the control mechanism module, a sound-permeable outlet is provided in the housing of the control mechanism module, and a waterproof sound-permeable membrane is disposed between the sound-permeable outlet and the buzzer. Compared with the traditional technical solution in which the buzzer is entirely enclosed in the control mechanism module, because of the sound-permeable outlet, a less loud sound signal emitted from the buzzer would be enough to raise the user's attention, which reduces the energy consumption of the buzzer, thereby optimizing the power consumption configuration of the infusion device and saving production costs.

Furthermore, the adhesive patch includes a tape and a protective film. The surface of the tape is provided with dense ventilation holes arranged evenly, which makes the tape breathable, and can help the patient to discharge the water vapor and sweat secreted by the skin of the patient, and avoid the water vapor and sweat to soak and crease the patient's skin.

### BRIEF DESCRIPTION OF THE DRAWINGS

FIG. 1a and FIG. 1b are schematic top views of the drug infusion device according to two embodiments of the present invention.

FIG. 2a-FIG. 2d are schematic views of the control mechanism module according to an embodiment of the present invention.

FIG. 3a is a schematic view of the infusion mechanism module according to an embodiment of the present invention.

FIG. 3b is a side view of the assembly of the control mechanism module and the infusion mechanism module according to an embodiment of the present invention.

FIG. 3c is a schematic top view of the lower case of the infusion mechanism module according to an embodiment of the present invention.

FIG. 3d is a schematic top view of the lower case of the infusion mechanism module according to another embodiment of the present invention.

FIG. 4a and FIG. 4b are schematic views of the internal mechanism module of the infusion mechanism module according to an embodiment of the present invention, respectively.

FIG. 4c is a schematic view of the internal mechanism module of the infusion mechanism module according to another embodiment of the present invention.

FIG. 5 is a schematic view of the elastic conductor according to an embodiment of the present invention.

FIG. 6 is a schematic view of the conductive tower-spring according to an embodiment of the present invention.

FIG. 7 is a partially enlarged view of the part M portion in FIG. 4a according to an embodiment of the present invention.

FIG. 8a and FIG. 8b are schematic views of the driving wheel assembly and the frame before and after assembly according to the embodiment of the present application embodiment, respectively.

FIG. 8c is a schematic view of the shield according to an embodiment of the present invention.

FIG. 9a-FIG. 9d are schematic views of the movable block opened or closed according to an embodiment of the present invention, respectively.

FIG. 10a-FIG. 10c are schematic views of the elastic element and the driving wheel assembly according to the embodiment of the present application, respectively.

FIG. 10d is a partially enlarged view of the N portion in FIG. 10b according to an embodiment of the present invention.

### DETAILED DESCRIPTION

As mentioned above, the reliability of the electrical connection between the control mechanism module and the infusion mechanism module of the present drug infusion device is poor.

In order to solve this problem, the present invention provides a drug infusion device, the control mechanism module and the infusion mechanism module are electrically connected to each other by a plurality of first electrical contact and a plurality of second electrical contact that are press againsted each other, optimizing the circuit design and improving the reliability of the electrical connection. In particular, an end portion of the first electrical contact protruds from the surface of the control mechanism module in contact with the first electrical contact, so that when the control mechanism module and the infusion mechanism module are assembled, the first electrical contact and the second electrical contact may be contacted preferentially, further improving the reliability of the electrical connection between the control mechanism module and the infusion mechanism module.

Various exemplary embodiments of the present invention will now be described in detail regarding the figures. The relative arrangement of the components and the steps, numerical expressions and numerical values outlined in the embodiments are not construed as limiting the scope of the invention.

In addition, it should be understood that, for ease of description, the dimensions of the various components shown in the figures are not necessarily drawn in the actual scale relationship; for example, the thickness, width, length or distance of certain units may be exaggerated relative to other mechanism modules.

The following description of the exemplary embodiments is merely illustrative and does not limit its application or use to the invention. The techniques, methods, and devices are known to those of ordinary skill in the art and may not be discussed in detail. However, such techniques, methods, and devices should be considered as part of the specification.

It should be noted that similar reference numerals and letters indicate similar items in the following figures. Therefore, once an item is defined or illustrated in a drawing, it will not be discussed further in the following description of the drawings.

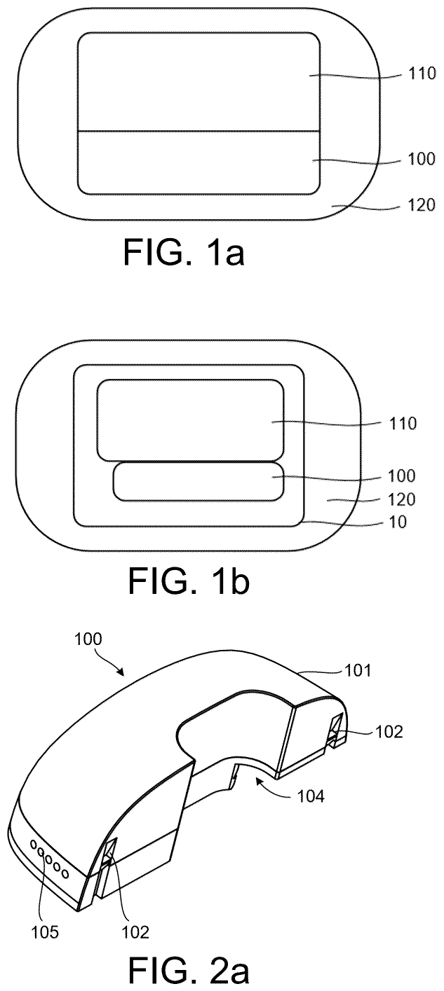

FIG. 1a and FIG. 1b are schematic top views of the drug infusion device according to two embodiments of the present invention.

In the embodiment of the present invention, the skin patch drug infusion device comprises a control mechanism module 100, an infusion mechanism module 110 and an adhesive patch 120, filling module 120, which will be described separately below. In other embodiments of the present invention, the patch-type drug infusion device may include more parts, which are not specifically limited here.

The patch-type drug infusion device refers to a tubing-free infusion device that is entirely pasted on the user's skin surface by the one piece of adhesive patch 120. And the infusion device is provided with an infusion needle unit 121, integrated on the infusion device, instead of a long tube; therefore, the drug can be directly infused from the drug reservoir 131 to the subcutaneous tissue through the infusion needle unit 121.

In the embodiment of the present invention, the infusion mechanism module 110 and the control mechanism module 100 are designed separately and connected by a waterproof plug or directly engaged and electrically connected into a whole. Details regarding how the reliability of the electrical connection has been improved when the infusion mechanism module 110 and the control mechanism module 100 are directly engaged and electrically connected into a whole will be described below. The infusion mechanism module 110 can be reused, and the control mechanism module 100 is discarded after a single use, as shown in FIG. 1a. In another embodiment of the present invention, the infusion mechanism module 110 and the control mechanism module 100 are connected by a wire and disposed of inside the same housing 10. Attached to a certain position of the user's skin by the adhesive patch 150, both units will be discarded together after a single use, as shown in FIG. 1b.

The patch-type drug infusion device of the embodiment of the present invention includes a control structure 100, which receives signals or information from a remote device or a body fluid parameter detection device (such as CGM), and controls the infusion device to infuse drug(s) accordingly.

Inside the housing 101 of the control mechanism module 100 are disposed of program modules, circuit board(s) and related electronic units for receiving signals or issuing control instructions, as well as other mechanical units or structures necessary for realizing the infusion function, which is not limited herein. In another embodiment of the present invention, a power supply can also be provided in the control mechanism module. Preferably, in the embodiment of the present invention, the power supply 133 is provided in the infusion mechanism module 110, which will be described below.

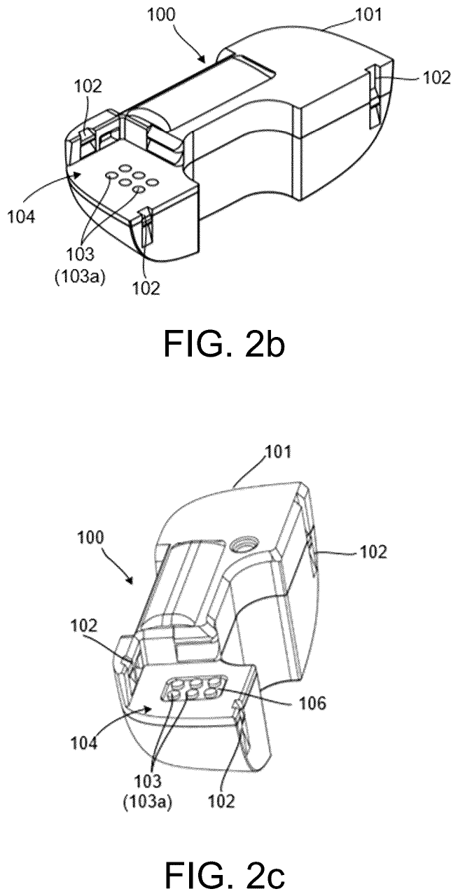

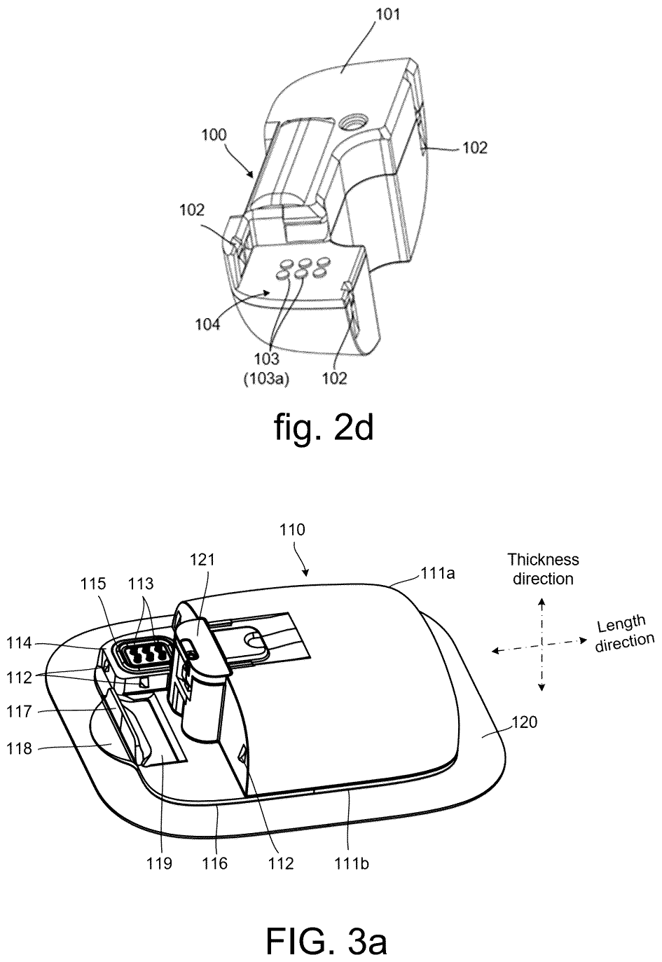

FIG. 2a-FIG. 2d are schematic views of the control mechanism module according to an embodiment of the present invention.

The control mechanism module 100 further includes the first electrical contact 103 exposed on its surface. The first electrical contact 103 is used as a circuit connection terminal to connect the internal circuits provided in the control mechanism module 100 and the infusion mechanism module 110, respectively. The embodiment of the present invention does not specifically limit the positions of the first electrical contact 103.

Compared with the plug connector used as a connection terminal in the prior arts, the contact area of the electrical contact is much smaller, which provides more flexibility to the mechanism module design, and can effectively reduce the size of the control mechanism module. At the same time, these smaller electrical contacts can be directly connected to the internal circuit or electrical components. They could also be directly soldered on the circuit board, which helps to optimize the design of the internal circuit and effectively reduce the complexity of the circuit, thereby saving costs and reducing the size of the infusion system. Furthermore, the electrical contacts are exposed on the surface of the control mechanism module 100 to facilitate electrical connection with connection ends on other mechanism modules. The above technical advantages of the electrical contacts apply to the first electrical contact 103 on the control mechanism module 100 and the second electrical mechanism module 113 on the infusion mechanism module 110, which will not be described in detail below.

The type of the first electrical contact 103 includes rigid metal pins or elastic conductive members. Preferably, in the embodiment of the present invention, the first electrical contact 103 is a rigid metal pin. One end of the first electrical contact 103 is electrically connected to the connection end provided inside the control mechanism module 100. In contrast, the other end is exposed on the surface of the housing 101. The rest part of the first electrical contact 103 is tightly embedded in the housing 101, thus keeping the internal control mechanism module 100 isolated from the outside. In the embodiment of the present invention, the control mechanism module 100 is further provided with a concave 104 for inter-assembling with the convex portion 114 at the bottom of the housing of the infusion mechanism module 110, as will be described in detail below. Specifically, the first electrical contact 103 is provided within the concave 104.

Here, the other end 103a of the first electrical contact 103 being exposed to the housing 101 comprises the other end 103a of the first electrical contact 103 being flush with the surface of the housing 101 as shown in FIG. 2b, and also comprises the other end 103a of the first electrical contact 103 protruding out of the surface of the housing 101 in contact with the first electrical contact 103, as shown in FIG. 2c and FIG. 2d. In FIG. 2c, a recess 106 is opened in the portion of the concave 104 surrounding the first electrical contact 103 such that the other end 103a of the first electrical contact 103 protrudes from the surface of the housing 101 in contact with the first electrical contact 103, i.e., the surface of the recess 106. In FIG. 2d, the other end 103a of the first electrical contact 103 is projected directly from the surface of the concave 104. By setting the other end 103a of the first electrical contact 103 to project on the surface of the housing 101 in contact with the first electrical contact 103, a reliable electrical connection between the control mechanism module 100 and the infusion mechanism module 110 can be realized when the control mechanism module 100 and the infusion mechanism module 110 are assembled, as will be described in detail below.

The elastic conductive member includes conductive spring, conductive silica gel, conductive rubber, or conductive leaf spring. One end of the elastic conductive member is used to electrically connect with the internal connection end in the control mechanism module 100, while the other end is used to connect with other connection ends electrically.

As in an embodiment of the present invention, the first electrical contact 103 is a conductive spring. When the electrical contacts are in contact with each other, the elasticity of the conductive spring can enhance the reliability of the electrical connection. Similar to the rigid metal pin, one end of the conductive spring is exposed on the surface of the housing 101. In contrast, the remaining part of the conductive spring is tightly embedded in the housing 101 and electrically connected with internal circuits or electrical components. The connection end disposed inside the control mechanism module 100 can be a conductive lead, a specific part of a circuit, or an electrical element.

It should be noted that the “tightly embedded” in the embodiment of the present invention suggests that there is no gap between the electrical contact and the housing 101, keeping the control mechanism module 100 tightly sealed. The following “tightly embedded” has the same meaning as here.

In another embodiment of the present invention, the first electrical contact 103 is a conductive spring, but it is not tightly embedded in the housing 101. Instead, a sealing element is provided in a groove, both of which are disposed around the area where the first electrical contacts 103 are located, thus sealing the electrical contact area and the control mechanism module 100.

In the embodiment of the present invention, the control mechanism module 100 is further provided with the first engaging portions 102, which is used to fasten the second engaging portion 112 disposed on the infusion mechanism module 110 to assemble the control mechanism module 100 infusion mechanism module 110. Details regarding how the mechanism works to enable the electrical connection between the first electrical contacts 103 and the second electrical contacts 113 will be described below.

The first engaging portion 102 and the second engaging portion 112 include one or more hooks, blocks, holes, and slots that can be engaged with each other. The positions of the hooks, blocks, holes, and slots can be flexibly adjusted, according to the shape and mechanism module features of the control mechanism module 100 and the infusion mechanism module 110, such as disposed in the interior or on the surface of the corresponding mechanism module, which is not specifically limited herein.

In the embodiment of the present invention, a buzzer (not shown) is also provided in the control mechanism module 100. When the infusion process starts or ends, the infusion device malfunctions, the drug is exhausted, the control mechanism module 100 issues an error command or receives an error message, etc., the buzzer is used to issue alarm signals, such as sound or vibration, notifying the user to adjust or replace the device in time.

Preferably, in the embodiment of the present invention, the housing 101 of the control mechanism module 100 is provided with a sound-permeable outlet 105 to allow the sound alarm signal from the buzzer to be sent out. In order to achieve a good sealing effect and ensure the normal operation of the buzzer, a waterproof sound-permeable membrane (not shown) is disposed between the sound-permeable outlet 105 and the buzzer. Therefore, the waterproof sound-permeable membrane needs to have a certain porosity to ensure the sound transmission but prevent water molecules penetration.

Compared with the traditional technical solution in which the buzzer is entirely enclosed in the control structure 100, because of the sound-permeable outlet 105, a less loud sound signal emitted from the buzzer would be enough to raise the user's attention, which reduces the energy consumption of the buzzer, thereby optimizing the power consumption configuration of the infusion device and saving production costs.

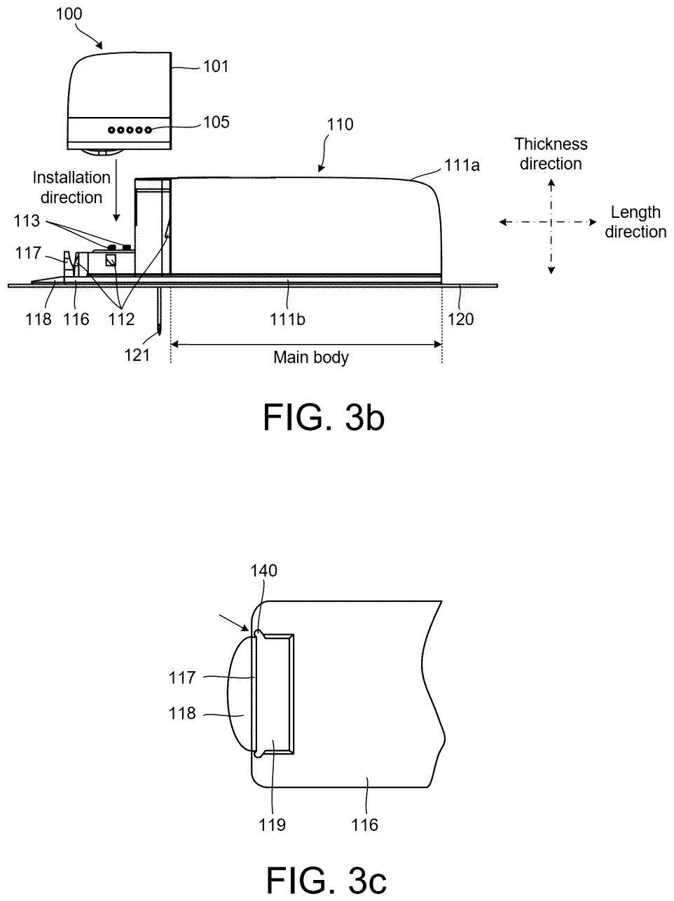

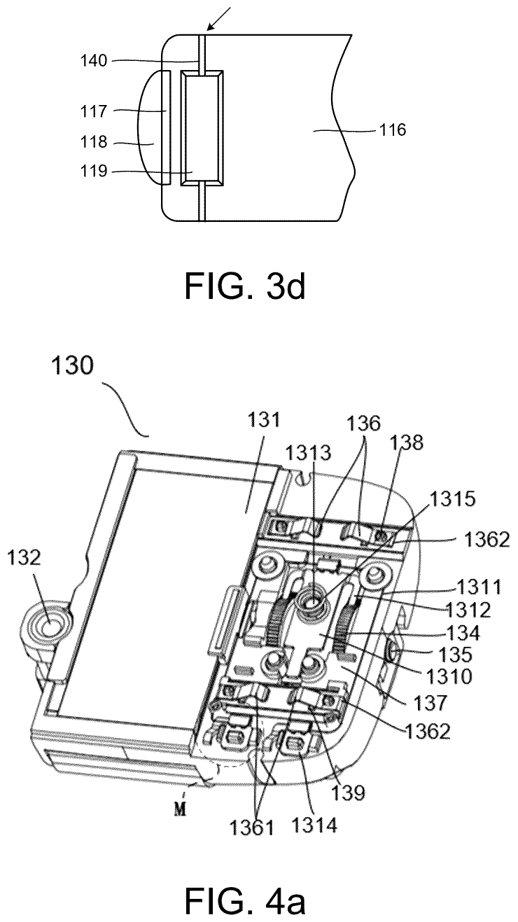

FIG. 3a is a schematic view of the infusion mechanism module 110 according to the embodiment of the present invention. FIG. 3b is a side view of the assembly of the control mechanism module 100 and the infusion mechanism module 110 according to the embodiment of the present invention. FIG. 3c is a schematic top view of the lower case of the infusion mechanism module according to an embodiment of the present invention. FIG. 3d is a schematic top view of the lower case of the infusion mechanism module according to another embodiment of the present invention.

The skin patch drug infusion device further includes an infusion mechanism module 110 with a case. A mechanical module, an electric control module, and other auxiliary modules for completing the drug infusion process are provided inside the case, which will be described in detail below. The case of the infusion mechanism module 110 may include multiple parts. As in the embodiment of the present invention, the case of the infusion system includes an upper case 111a and a lower case 111b.

As mentioned above, in the embodiment of the present invention, the infusion mechanism module 110 is provided with the second engaging portions 112, which is used to engaged and fasten the corresponding first engaging portions 102. The positions where the first engaging portions 102 and the second engaging portions 112 are provided correspondingly.

In the embodiment of the present invention, the infusion mechanism module 110 is provided with second electrical contacts 113, which are used to press against the corresponding first electrical contacts 103 to create an electrical connection between the control mechanism module 100 and the infusion mechanism module 110. The compression between these two corresponding electrical contacts disposed on different structures can improve the reliability of the electrical connection. Similar to the first electrical contacts 103, one of the second electrical contact 113 also includes a rigid metal pin and an elastic conductive member. Preferably, in the embodiment of the present invention, the second electrical contact 113 is a conductive spring. Similarly, the conductive spring can improve the electrical connection performance. A groove is also arranged around the area where the second electrical contact 113 is disposed, and a sealing member 115 is arranged in the groove. Similarly, the elasticity of the conductive spring can further improve the electrical connection performance.

Preferably, in the embodiment of the present invention, the two ends of the conductive spring have different diameters. And the diameter of the end exposed to the outside of the infusion mechanism module 110 is shorter than that of the end inside the infusion mechanism module 110. In this way, the conductive spring can be held in the case because of the longer diameter; Thus, when the control mechanism module 100 is not installed on the infusion mechanism module 110, the longer diameter of the inner end can prevent the conductive spring from detaching from the infusion mechanism module 110.

The embodiment of the present invention does not limit the position at where second electrical contacts 113 are arranged, as long as it can be electrically connected to the corresponding first electrical contacts 103. Preferably, in the embodiment of the present invention, the upper case 111a of the infusion mechanism module 110 includes a convex portion 114 where the second electrical contacts 113 are disposed, as shown in FIG. 3a. The convex portion 114 corresponds to the concave 104 disposed on the control mechanism module 100, the two portions can be assembled to make the first electrical contacts 103 and the corresponding second electrical contacts 113 against each other to realize the electrical connection. When the other end 103a of the first electrical contact 103 protrudes from the surface of the housing 101 in contact with the first electrical contact 103, as shown in FIG. 2c and FIG. 2d, the first electrical contact 103 and the corresponding second electrical contact 113 may be preferentially contacted when the convex portion 114 is assembled with the concave 104 on the control mechanism module 100, which can avoide that the second electrical contact 113 is tilted during the assembly process, so that the second electrical contact 113 preferentially contacts the surface of the concave 104, resulting in the first electrical contact 103 and the second electrical contact 113 not being able to be completely press againsted ed, which in turn makes the electrical connection between the control mechanism module 100 and the infusion mechanism module 110 less reliable.

When the other end 103a of the first electrical contact 103 protrudes directly from the surface of the concave 104, as shown in FIG. 2d, in order to avoid interference between the control mechanism module 100 and the infusion mechanism module 110 during assembly, a second groove (not shown) is provided on the convex portion 114 of the infusio mechanism module 110 adjacent to the second electrical contact 113.

In one embodiment of the present invention, the first electrical contact 103 is a rigid metal contact, and the other end 103a exposed to the surface of the housing 101 is provided with an arcuate end, and the second electrical contact 113 is an electrically conductive spring, so that the first electrical contact 103 with an arcuate end can be inserted into the second electrical contact 113, i.e., the inner cavity of the electrically conductive spring, during the assembly process of the control mechanism module 100 and the infusion mechanism module 110, so as to make the connection of the first electrical contact 103 and the second electrical contact 113 more stable, and thus to enhance the reliability of the electrical connection of the control mechanism module 100 and the infusion mechanism module 110.

Here, the meaning of the other end 103a of the first electrical contact 103 being exposed to the surface of the housing 101 is the same as before, i.e., it includes the other end 103a of the first electrical contact 103 being flush with the surface of the housing 101 as shown in FIG. 2b, and it also includes the other end 103a of the first electrical contact 103 protruding out of the surface of the housing 101 that is in contact with the first electrical contact 103, as shown in FIG. 2c and FIG. 2d.

In other embodiments of the present invention, the convex portion 114 may be provided on the lower case 111b. When the infusion mechanism module 110 includes an integral case, the convex portion 114 will be a part of the integral case not specifically limited herein.

The method of assembling the control mechanism module 100 and the infusion mechanism module 110 to each other includes pressing the control mechanism module 100 on the infusion mechanism module 110 along the thickness direction of the infusion mechanism module 110, thereby engaging the first engaging portion 102 and the second engaging portion 112; or pressing the control mechanism module 100 on the infusion mechanism module 110 along the length direction of the infusion mechanism module 110. Alternatively, the control mechanism module 100 can be pressed along with any angle between the thickness direction and the length direction of the infusion mechanism module 110, making the first engaging portion 102 and the second engaging portion 112 engaged with each other. Preferably, in the implementation of the present invention, the method by which the control mechanism module 100 and the infusion mechanism module 110 are assembled is to press the control mechanism module 100 on the infusion mechanism module 110 along with the thickness direction of the infusion mechanism module 110, making the first engaging portion 102 and the second engaging portion 112 engaged with each other, as shown the installation direction in FIG. 3b.

In the embodiment of the present invention, the lower case 111b of the infusion mechanism module 110 further includes an outward extending portion 116. A block 117 is provided outside the outer extending portion 116, as shown in FIG. 3a. As mentioned above, the control mechanism module 100 is pressed to the engaging position along the thickness direction of the infusion mechanism module 110; thus, block 117 can prevent the control mechanism module 100 from detaching along the length direction of the infusion mechanism module 110, ensuring the normal operation of the infusion system. Obviously, in another embodiment of the present invention, if the control mechanism module 100 is pressed to the engaging position along with other directions, the control mechanism module 100 can also be prevented from detaching from the infusion mechanism module 110 by adjusting the position of the block 117.

It should be noted here that “outer” and “outside” are relative to the main body of the infusion mechanism module 110, where they belong to a concept of the relative position, as shown in FIG. 3a or FIG. 3b. The “outside” below have the same meaning as here.

In the embodiment of the present invention, the outer end of the outer extending portion 116 is also provided with a pressing portion 118 for releasing the blocking effect of block 117. While the user is replacing the infusion mechanism module 110, the control mechanism module 100 can be released from block 117 by pressing the pressing portion 118 with a finger. Then, the user can remove the control mechanism module 100 from the infusion mechanism module 110 with two fingers.

Another embodiment of the present invention can also be provided with an unlocking hole 119 disposed of in the inner side of block 117. While the pressing portion 118 is pressed, a finger can enter the unlocking hole 119, thereby pushing the control mechanism module 100 out to separate the control mechanism module 100 from the infusion mechanism module 110. In the embodiment of the present invention, the unlocking hole 119 is square. The square unlocking hole 119 can facilitate the smooth entry of fingers. In other embodiments of the present invention, the unlocking hole 119 may also have other shapes, which is not specifically limited here.

The lower case 111b of the infusion mechanism module 110 is also provided with one or more crease grooves 140. Two crease grooves 140 are provided on both sides of the unlocking hole 119, as shown in FIG. 3c and FIG. 3d. After the crease groove, 140 is provided, the thickness or width of the lower case 111b at the crease groove 140 (as shown by the arrows in FIG. 3c and FIG. 3d) is reduced. When the user presses the pressing portion 118, the lower case 111b is easily broken at the crease groove 140, and the blocking of the control mechanism module 100 by block 117 is more smoothly released.

Preferably, in the embodiment of the present invention, two crease grooves 140 are provided at the both ends of block 117, respectively, as shown in FIG. 3c. In another embodiment of the present invention, the crease groove 140 is provided on two corresponding lateral sides of the unlocking hole 119, as shown in FIG. 3d.

The skin patch drug infusion device further includes a needle unit 121, used for infusing the drug to the subcutaneous tissue.

An adhesive patch 120 is also provided on the bottom of the lower case 111b to attach the infusion device to the user's skin surface. The adhesive patch includes a tape and a protective film. The surface of the tape is provided with dense ventilation holes arranged evenly, which makes the tape breathable, and can help the patient to discharge the water vapor and sweat secreted by the skin of the patient, and avoid the water vapor and sweat to soak and crease the patient's skin.

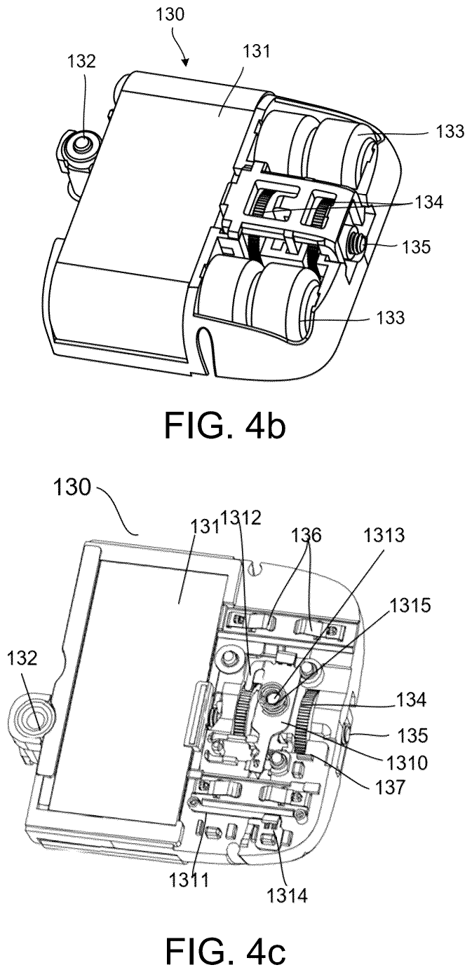

FIG. 4a and FIG. 4b are two schematic views of the internal mechanism module 130 of the infusion mechanism module 110 of the embodiment of the present invention from two perspectives, respectively. FIG. 4c is a schematic view of the internal mechanism module 130 of the infusion mechanism module according to another embodiment of the present invention.

In the embodiment of the present invention, the internal mechanism module 130 includes mechanical units and electronic control units used to realize the infusion function, such as a drug reservoir 131, a drug outlet 132, a power supply 133, a driving wheel 134, a screw 135, a circuit board (not shown), a driving unit 1310, etc. The movement of the driving unit 1310 drives the driving wheel 134 to rotate, thus making the screw 135 push the piston (not shown) in the drug reservoir 131 forward, realizing the drug infusion.

In the embodiment of the present invention, the power supply 133 is a conventional button battery. In other embodiments of the present invention, the power supply 133 may also be other types of batteries, as long as it can meet the requirements for supplying power to the infusion system. Preferably, in the embodiment of this present invention, the type of the power supply 133 is a double-row battery pack; that is, two rows of button batteries are arranged on both sides of the driving wheel 134, respectively, as shown in FIG. 4b. Conventionally, the discharge capacity of button batteries is low. The double-row button battery pack can reduce the discharge level of each battery, thereby extending the service life of the battery. Furthermore, the double-row design of the power supply 133 can make the full use of the internal space and improve the integration within the internal mechanism module in the infusion system.

The infusion mechanism module 110 in the embodiment of the present invention is also provided with a circuit board or multiple three-dimensional circuits coated on the surface of a part of the mechanism module for supplying power to specific structural units. According to the internal arrangement characteristics of the infusion device, the shape and position of the three-dimensional circuit can be flexibly designed, which can make the full use of the internal space of the infusion mechanism module, making the arrangement more compact. The circuit board is a hard/rigid circuit board or a flexible circuit board. Preferably, in the embodiment of the present invention, the circuit board is flexible. The shape of the flexible circuit board is adjustable, allowing it to be flexibly designed according to the internal space of the infusion mechanism module 110. At the same time, multiple connection ends can be provided on the flexible circuit board to be electrically connected to second electrical contacts 113, thereby connecting the circuits of the control mechanism module 100 and the infusion mechanism module 110, allowing the infusion system to perform drug infusion function.

An elastic conductor 136 is also provided inside the infusion mechanism module 130. The elastic conductor 136 is electrically connected to the power supply 133, and the specific connection end on the circuit board (or three-dimensional circuit), thereby supplying power to specific structural units.

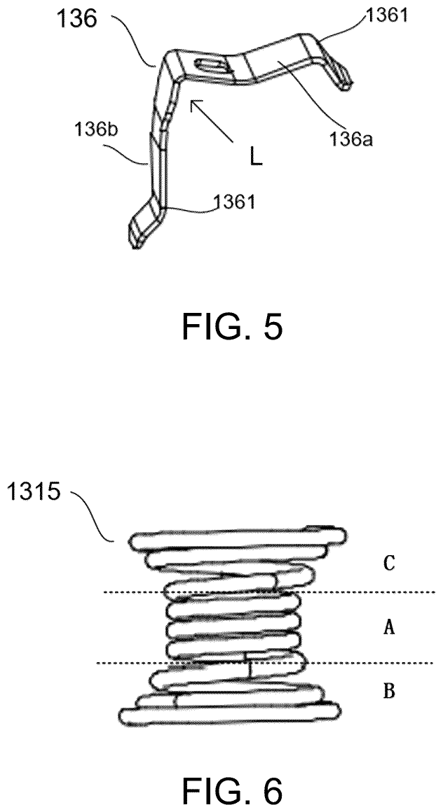

FIG. 5 is a schematic view of the elastic conductor according to an embodiment of the present invention.

In the embodiment of the present invention, the elastic conductor 136 includes a first elastic conductor 136a and a second elastic conductor 136b, the first elastic conductor 136a is connected with the power supply 133, and the second elastic conductor 136b is connected with the specific connection end on the circuit board (or three-dimensional circuit). At least a protrusion 1361 is provided on the first elastic conductor 136a and the second elastic conductor 136b, which facilitate the point contact connection or the line contact connection between the first elastic conductor 136a and the power supply 133, and also facilitate the point contact connection or the line contact connection between the second elastic conductor 136b and the specific connection end on the circuit board (or three-dimensional circuit), thereby improving the electrical connection reliability between the elastic conductor 136 and the power supply 133, and the specific connection end on the circuit board (or three-dimensional circuit). When the first elastic conductor 136a and the second elastic conductor 136b is flat, during use, it is likely to cause a poor connection between the elastic conductor 136 and the power supply 133 and the specific connection end on the circuit board (or three-dimensional circuit), thereby affecting the use effect. In the embodiment of the present invention, protrusion 1361 may be a linear protrusion formed by bending the first elastic conductor 136a or the second elastic conductor 136b, or it may be several dots or other shapes of protrusions formed by other means on the first elastic conductor 136a or the second elastic conductor 136b. The form and number of the protrusions 1361 on the first elastic conductor 136a or the second elastic conductor 136b can be the same or different. Here, the form, number, and forming method of the protrusions 1361 are not specifically limited, as long as the point contact connection or line contact connection between the elastic conductor 136 and the power supply 133 and the specific connection end on the circuit board (or three-dimensional circuit) can be enhanced, improving the reliability of the electrical connection.

In the embodiment of the present invention, the first elastic conductor 136a and the second elastic conductor 136b are approximately arranged in an “L” shape, the first elastic conductor 136a is approximately parallel to the frame 137, and the second elastic conductor 136b is approximately perpendicular to the frame 137. In other embodiments of the present invention, the first elastic conductor 136a and the second elastic conductor 136b can also be arranged in other shapes, which are not specifically limited here, as long as the power supply 133 and the specific connection end on the circuit board (or three-dimensional circuit) can be electrically connected. An insulating member 1362 is also provided at the junction of the first elastic conductor 136a and the second elastic conductor 136b (as shown at the position L in FIG. 5) to prevent the power unit 1311 from contacting the elastic conductor 136 and causing a short circuit during operation, further stop the infusion mechanism module 130 from working. In the embodiment of the present invention, the insulating member 1362 is formed by printing ink. In other embodiments of the present invention, the insulating member 1362 may also be insulating glue, insulating varnish or insulating material, which is not specifically limited herein.

In the embodiment of the present invention, the elastic conductor 136 may be a sheet metal including the first elastic conductor 136a and the second elastic conductor 136b or may be integrated by the single first elastic conductor 136a and the single second elastic conductor 136b which are directly electrically connected or indirectly electrically connected with other conductive elements, which is not limited herein. When the elastic conductor 136 is a sheet metal including the first elastic conductor 136a and the second elastic conductor 136b, which not only make the elastic conductor 136 with simple technological process, firm connection between the elastic conductor 136a and the second elastic conductor 136b, but also reduce the volume of the elastic conductor 136, save the material and the the raw material and technological cost. When the elastic conductor 136 is integrated by directly electrically connecting the first elastic conductor 136a and the second elastic conductor 136b or indirectly electrically connected with other conductive elements, the elastic member can be flexibly selected according to the requirements of the specific connecting components to optimize the internal design of the infusion mechanism module.

In the embodiment of the present invention, the frame 137 is further provided with a positioning post 138, the elastic conductor 136 is provided with an opening corresponding to the positioning post 138, and the elastic conductor 136 is sleeved on the positioning post 138 through the opening, so that the elastic conductor 136 is fixed to the frame 137. At the same time, the positioning post 138 is melted by hot melting to further fixed the elastic conductor 136 and to prevent the elastic conductor 136 from shaking due to long-term use or other reasons, which cause the poor electric connection between the elastic conductor 136 and the power supply 133 and the specific connection end on the circuit board (or three-dimensional circuit) and affect the use.

In the embodiment of the present invention, a boss 139 is also provided on frame 137. The boss 139 is located below the first elastic conductor 136a to prevent the protrusion 1361 of the first elastic conductor 136a from being flattened or deformed during long-term use, which results in a poor electrical connection between the first elastic conductor 136a and the specific connection end on the circuit board (or three-dimensional circuit), causing a malfunction. Preferably, the setting position of the boss 139 deviates from the projection position of the protrusion 1361 on the frame 137 and is close to the position post 138. On the one hand, it can prevent the circuit board from being damaged by hard contact or compression between the first elastic conductor 136a and the specific connection end on the circuit board (or three-dimensional circuit). On the other hand, it can ensure the elastic electrical contact between the first elastic conductor 136a and the specific connection end on the circuit board (or three-dimensional circuit).

Similar to the elastic conductive member above mentioned, the type of the elastic conductor 136 includes a conductive spring, a conductive leaf spring, a conductive rubber, a conductive silica gel, etc., which are not specifically limited herein, as long as they can meet the requirements for electrically connecting the power supply 133 to specific connection ends on the circuit board (or three-dimensional circuit). Preferably, in the embodiment of the present invention, the elastic conductor 136 is the conductive leaf spring. Since the infusion mechanism module 110 has a double-row battery pack, the multiple conductive leaf springs are also designed as a double-row pack, as shown in FIG. 4a.

As shown in FIG. 4a, the interior of the infusion mechanism module 110 also includes a drive unit 1310 and a power unit 1311, arranged on the frame 137. Frame 137 is also provided with a rotation shaft 1313, the drive unit 1310 is provided with a through hole, and the drive unit 1310 is sleeved on the rotation shaft 1313 through a through hole for fixing. The elastic conductor 136 can realize a direct electrical connection between the power supply 133 and the specific structural units, which helps to optimize the internal circuit design and reduce the complexity of the internal mechanism module. The driving unit 1310 includes a driving end 1312. One end of the power unit 1311 is connected to the driving unit 1310, and the other end is connected to a specific connection ends on the circuit board or three-dimensional circuit through the conductive platform 1314 so as to further realize the connection with the control module in the control mechanism module 100. The control module in the control mechanism module 100 applies power to the power unit 1311, the drive unit 1310 rotates around the rotation shaft 1313, drives the drive end 1312 forward to push the gear teeth of the drive wheel 134 forward, and causes the infusion device to perform drug delivery. In the embodiment of the present invention, there are two driving ends 1312 and two corresponding power units 1311. In another embodiment of the present invention, there is one driving end 1312 and one power unit 1311, as shown in FIG. 4c.

The infusion mechanism module 110 is also provided with a conductive tower-spring 1315 sleeved on the rotating shaft 1313, with one end abuts the drive unit 1310, and the other end electrically connected to a specific connection end on the circuit board or three-dimensional circuit for fixing the drive unit 1310, at the same time, realize the electrical connection between the driving unit 1310 and specific connection ends on the circuit board or three-dimensional circuit.

FIG. 6 is a schematic view of the conductive tower-spring according to an embodiment of the present invention.

In the embodiment of the present invention, the conductive tower spring 1315 includes a part A with a small diameter at the central part in the axial direction and a part B and a part C with a large diameter at both ends. The diameter of part A remains the same, and the rotation shaft 1313 is fixed by interference fit, further, fix the drive unit 1310, and at the same time, part B abuts the drive unit 1310 to prevent the drive unit 1310 from shaking due to instability when it is accidentally touched, causing the drive end 1312 to push the drive wheel 134 forward, which affects the accuracy of the infusion. The diameter of the B part and the C part gradually expand towards the two ends in a horn-like shape. When the B part and the C part are respectively electrically connected to the drive unit 1310 and the specific connection ends on the circuit board or three-dimensional circuit, the conductive tower-spring 1315 is compressed; therefore, part B and part C have multiple turns, and at least 2-3 turns of springs are in contact with the driving unit 1310 and the specific connection ends on the circuit board or three-dimensional circuit at the same time, thereby improving the electrical connection reliability of the infusion device. Preferably, in the embodiment of the present invention, parts B and C of the conductive tower-spring 1315 are symmetrical, and the two ends can be assembled at will to avoid assembly misalignment.

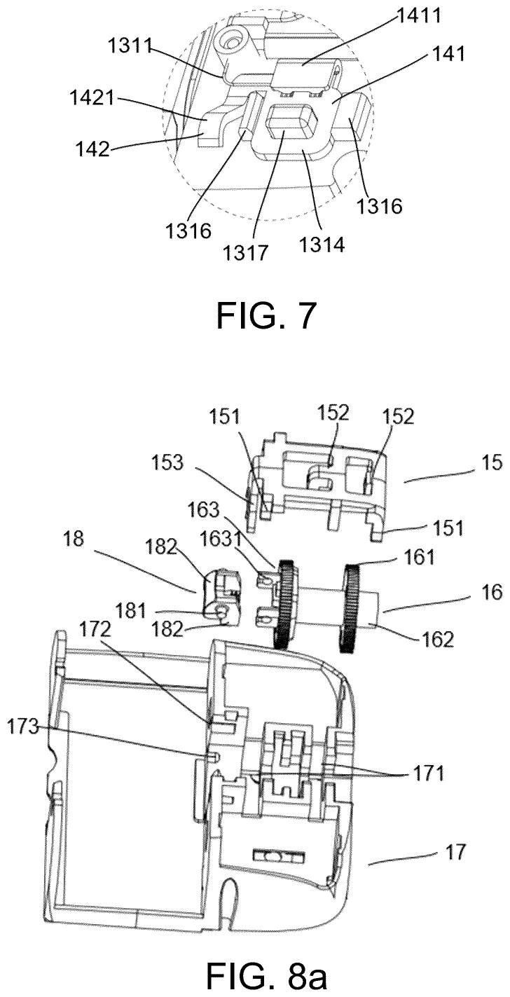

FIG. 7 is a partially enlarged view of the part M portion in FIG. 4a according to the embodiment of the present invention.

In the embodiment of the present invention, at least one conductive platform 1314 is provided inside the infusion mechanism module 110. Specifically, when the driving unit 1310 includes two driving arms 1312, there are two corresponding power units 1311 and conductive platforms 1314, as shown in FIG. 4a. When the driving unit 1310 includes one driving arm 1312, there is one corresponding power unit 1311 and one conductive platform 1314, as shown in FIG. 4c. The conductive platform 1314 is electrically connected to the power unit 1311 and the specific terminal on the circuit board or three-dimensional circuit, respectively.

The conductive platform 1314 includes a conductive platform body 141 and a conductive arm 142. The conductive arm 142 is an elastic conductive element. At least one conductive platform protrusion 1421 is provided on the conductive arm 142 to facilitate the connection with specific connection ends on the circuit board (or three-dimensional circuit), improving the electrical connection reliability of the conductive platform 1314 and the specific connection ends on the circuit board (or three-dimensional circuit). When the conductive arm 142 of the conductive platform 1314 is flat, it is likely to cause the poor connection between the conductive platform 1314 and specific connection ends on the circuit board (or three-dimensional circuit) during use, thereby affecting the use effect. In the embodiment of the present invention, conductive platform protrusion 1421 may be a linear protrusion formed by bending conductive arm 142, or it may be several dots or other shapes of protrusions formed by other means on conductive arm 142. Here, the form, number, and forming method of conductive platform protrusion 1421 are not specifically limited, as long as the point contact connection or line contact connection between the conductive platform 1314 and the specific connection ends on the circuit board (or three-dimensional circuit) can be enhanced, improving the reliability of the electrical connection. In the embodiment of the present invention, the conductive platform body 141 further includes an end portion 1411 of the conductive platform body, and the power unit 1311 is an electric drive type linear driver or an electric heating type linear driver, such as a shape memory alloy, which is connected to the conductive platform body 141 by die casting, causing stable connection and highly reliable electrical connection. Specifically, the power unit 1311 is put into the end portion 1411 of the conductive platform body after being folded in half and then connected to the end portion 1411 of the conductive platform body 141 by die-casting method further improving the reliability of the electrical connection.

In the embodiment of the present invention, the conductive platform 1314 may be a sheet metal including the conductive platform body 141 and conductive arm 142, or may be formed by the single conductive platform body 141 and the single conductive arm 142 directly electrically connected or indirectly electrically connected with other conductive elements, which is not limited herein. When the conductive platform 1314 is a sheet metal including the conductive platform body 141 and the conductive arm 142, which not only make the conductive platform 1314 with simple technological process, firm connection between the conductive platform body 141 and conductive arm 142, but also reduce the volume of the conductive platform 1314, save the material and the the raw material and technological cost. When the conductive platform 1314 is integrated by the single conductive platform body 141 and the single conductive arm 142 directly electrically connected or indirectly connected with other conductive elements, the conductive member can be flexibly selected according to the requirements of the specific connecting components to optimize the internal design of the infusion mechanism module.

Similarly, the conductive platform body 141 and the conductive platform body end portion 1411 can also be sheet metal or integrated by directly or indirectly electrically connecting with other conductive elements, which is not limited herein. The conductive platform 1314 is a sheet metal including the conductive platform body 141, the conductive arm 142 and the conductive platform body end portion 1411, or the two of them can be a sheet metal and then integrated with the third party directly or indirectly through other conductive elements, or all of the three parts are directly integrated or indirectly integrated by electrical connection with other conductive elements, and there is no specific limitation here. The benefits of various forming methods are described above, so it will not be repeated here. Preferably, in the embodiment of the present invention, the conductive platform body 141, the conductive arm 142 and the conductive platform body end portion 1411 are integrally formed.

In the embodiment of the present invention, a plurality of stoppers 1316 are provided on the frame 137 for accommodating and limiting the position of the conductive platform 1314, the frame 137 is further provided with a conductive platform positioning post 1317, the conductive platform 1314 is provided with an opening corresponding to the conductive platform positioning post 1317, and the conductive platform 1314 is sleeved on the conductive platform positioning post 1317 through the opening, so that the conductive platform 1314 is fixed to the frame 137. At the same time, the conductive platform positioning post 1317 is melted by hot melting to further fixed the conductive platform 1314 and to prevent the conductive platform 1314 from shaking due to long-term use or other reasons, which cause the poor electric connection between the conductive platform 1314 and the power supply 133 and the specific connection end on the circuit board (or three-dimensional circuit) and affect the use.

In the embodiment of the present invention, a conductive platform boss (not shown) is also provided on the frame 137, and the conductive platform boss is located below the conductive arm 142 to prevent the conductive platform protrusion 1421 of the conductive arm 142 from being flattened or deformed during long-term use, which results in a poor electrical connection between the conductive arm 142 and the specific connection end on the circuit board (or three-dimensional circuit), causing a malfunction. Preferably, the setting position of the conductive platform boss deviates from the projection position of the conductive platform protrusion 1421 on the frame 137 and it is close to the conductive platform positioning post 1317. On the one hand, it can prevent the circuit board from being damaged by hard contact or compression between the conductive arm 142 and the specific connection end on the circuit board (or three-dimensional circuit). On the other hand, it can ensure the elastic electrical contact between the conductive arm 142 and the specific connection end on the circuit board (or three-dimensional circuit).

Similar to the elastic conductor 136 above mentioned, the type of the conductive platform 1314 includes a conductive spring, a conductive leaf spring, a conductive rubber, a conductive silica gel, etc., which are not specifically limited herein, as long as they can meet the requirements for electrically connecting the power unit 1310 to specific connection ends on the circuit board (or three-dimensional circuit).

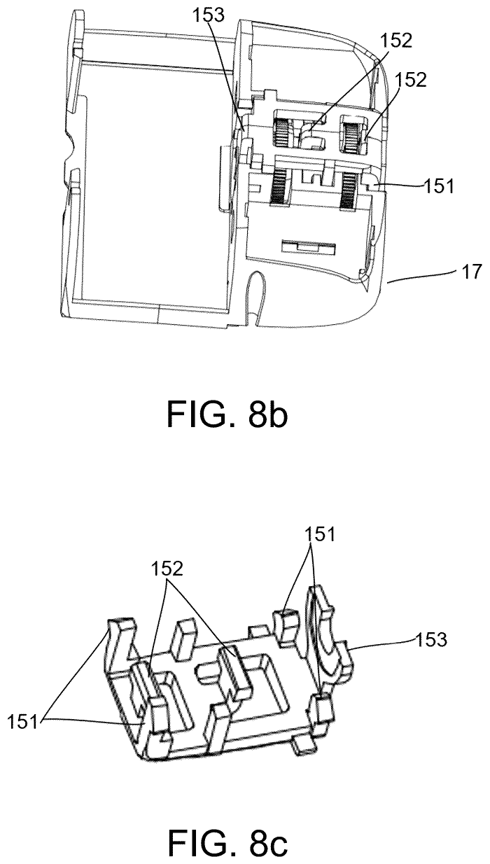

FIG. 8a and FIG. 8b are schematic views of the driving wheel assembly and the frame before and after assembly according to the embodiment of the present application embodiment, respectively. FIG. 8c is a schematic view of the shield according to an embodiment of the present invention.

The driving wheel assembly includes a driving wheel body 16 and a movable block 18. The driving wheel body 16 includes a driving wheel 161, a driving tube 162, which is provided with threads for accommodating the screw 135, and a connecting piece 163 arranged at the end of the driving wheel 161. The connecting piece 163 is provided with a connecting portion 1631, operatively connected with the movable block 18. The movable block 18 is provided with threads to accommodate the screw 135. The movable block 18 is also provided with a movable block connecting rod 181, which is mobily connected with the connecting portion 1631. After the connecting rod 181 is mobily connected with the connecting portion 1631, the movable block 18 can rotate around the connecting rod 181 to open or close the movable block 18. When the movable block 18 is opened, the screw 135 can slide to the driving wheel body 16, and when the movable block 18 is closed, the screw 135 cannot slide to the driving wheel body 16, which will be described in detail below.

The driving wheel assembly further includes a shield 15, and a plurality of shield engaging portions 151 are provided on the shield 15 for engaging with the frame 17. The frame 17 is provided with a frame groove 171 for accommodating the driving wheel body 16 and a plurality of frame engaging portions 172 for engaging with the shield engaging portions 151. After the driving wheel body 16 and the movable block 18 are connected and put into the frame groove 171, the shield 15 is assembled to the frame 17. By the engagement of the frame engaging portion 173 and the shield engaging portion 151, the driving wheel body 16 and the movable block 18 are fixed in the frame 17 to prevent the shaking of the driving wheel assembly and affect the accuracy of drug infusion.

The frame engaging portions 173 and the shield engaging portion 151 include hooks, blocks, holes, and grooves that can be engaged with each other. The position of the engaging portions can be flexibly designed according to the shape and design of the frame 17 and the shield 15, and is not specifically limited here. Preferably, the space formed by the plurality of frame engaging portions 173 and the plurality of shield engaging portions 151 can accommodate the driving wheel body 16 and the movable block 18 to prevent the driving wheel body 16 from shaking.

In the embodiment of the present invention, the shield 15 is also provided with at least one elastic arm 152 for elastically abutting the drive tube 162, which can further fix the drive wheel body 16, improving the stability of the infusion mechanism module 110 and the accuracy of drug infusion. Preferably, two elastic arms 152 are arranged on both sides of one of the driving wheels 161 and abut against the driving tube 162, respectively. As shown in FIG. 8b, the driving wheel body 16 is fixed at multiple points.

The shield 15 is also provided with a baffle 153, and the baffle 153 is provided with a mouth. The main frame 17 is also provided with a card slot 173 on the side close to the reservoir 131 for accommodating the strip (not shown). The strip abuts against the baffle 153 to form a through hole for accommodating the screw 135 and at the same time confine the driving wheel body 16 and the movable block 18 in the frame 17. Preferably, the mouth of the baffle 153 is semi-circular, and the diameter is adapted to the diameter of the screw 135, so as to better accommodate the screw 135 and prevent the screw 135 from shaking greatly under the action of external force.

Preferably, in the embodiment of the present invention, the shield 15 is integrally molded; the technological process is simple, the the volume is small, the connection is firm, the material is saved, and the cost is reduced.

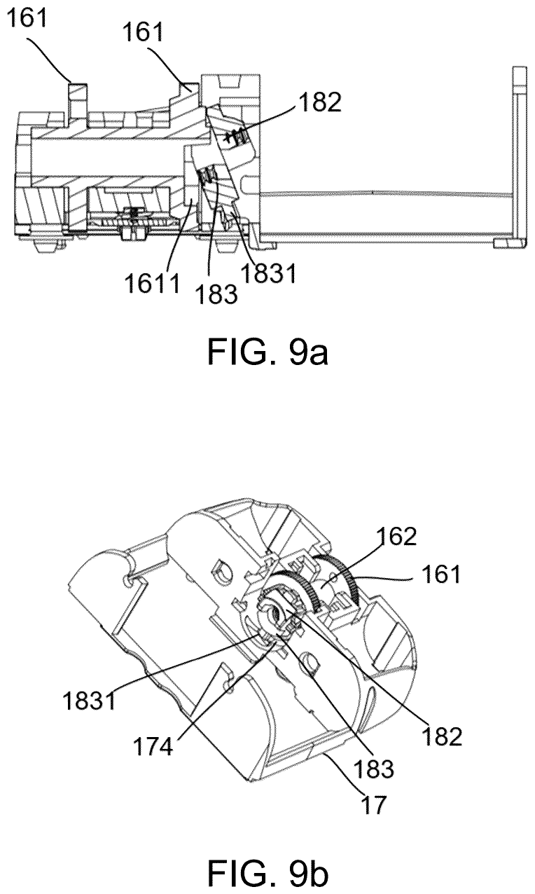

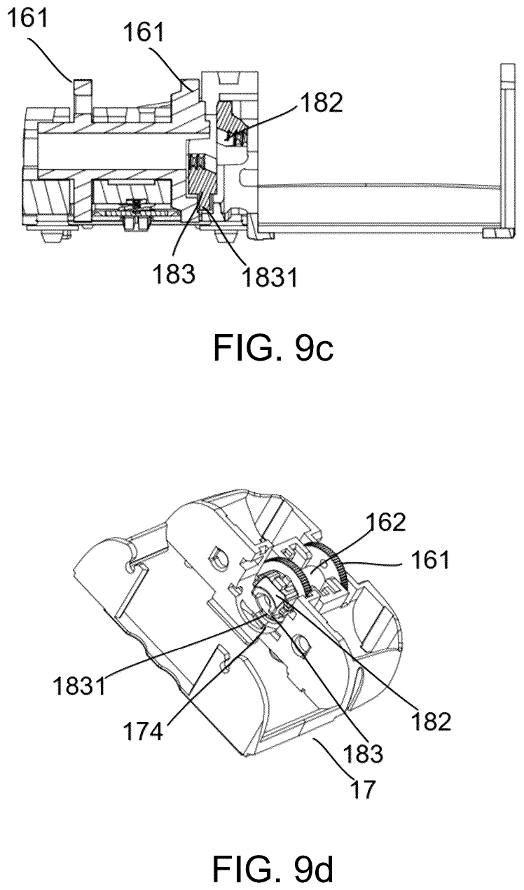

FIG. 9a-FIG. 9d are schematic views of the movable block opened or closed according to an embodiment of the present invention, respectively.

The movable block 18 further includes an upper movable block 182 and a lower movable block 183, and the lower movable block 183 is also provided with a lower movable block end 1831. The movable block 18 opened means that the upper movable block 182 is close to the driving wheel 161, and the lower movable block 183 is far away from the driving wheel 161. The thread in the movable block 18 does not engage with the screw 135. The screw 135 can be smoothly slid in the movable block 18 and the driving tube 162. The movable block 18 closed means that the upper movable block 182 is far away from the driving wheel 161, and the lower movable block 183 is close to the driving wheel 161. At this time, the thread in the movable block 18 is engaged with the screw rod 135, and the screw 135 cannot slide in the movable block 18 and the driving tube 162.

In the embodiment of the present invention, an arc-shaped blocking member 174 is also provided on the frame 17 to limit the position of the lower movable block 183. When the movable block 18 is opened, that is, the upper movable block 182 is close to the driving wheel 161 and the lower movable block 183 is far away from the driving wheel 161, the lower movable block end 1831 of the lower movable block 183 is located outside the arc-shaped blocking member 174, so that the movable block 183 is kept open. At this time, the thread in the movable block 18 does not engage with the screw 135, and the screw 135 can slide smoothly in the movable block 18 and the drive tube 162. When the movable block 18 is opened, the reservoir 131 can be filled with the drug. In the filling process to the reservoir 131, the screw 135 can smoothly move to the end of the driving wheel 161 under the pushing action generated during the filling process, until the filling is completed, no need for the assistance of other parts, which can simplify the complexity of the infusion mechanism module, improve the integration level, save cost, and reduce the volume of the infusion mechanism 110, and at the same time maximizing the actual storage volume of reservoir 131.

When the movable block 18 is closed, that is, the movable block 182 is far away from the driving wheel 161 and the lower movable block 183 is close to the driving wheel 161, the lower movable block end 1831 of the lower movable block 183 is located inside the arc-shaped blocking member 174, and the movable block 18 is kept closed. At this time, the thread in the movable block 18 is engaged with the screw 135, and the screw 135 cannot slide in the movable block 18 and the drive tube 162. When the infusion device 110 performs the drug infusion after the drug is filled, the driving end 1312 of the driving unit 1310 pushes the driving wheel 161 forward, and the movable block 18 rotates with the driving wheel 161. During the rotation, after the lower movable block end 1831 bypasses the arc-shaped blocking member 174, since there is no resistance from the arc-shaped blocking member 174, the lower movable block end 1831 falls into the inside of the arc-shaped blocking member 174. At the same time, since the movable block 18 is kept closed, the screw 135 can only move away from the driving wheel 161 under the pushing action of the rotation of the driving wheel 161 and perform the drug infusion. There is no need to worry about the free movement of the screw 135 due to the engagement failure of the screw 135 with the movable block 18 or the drive tube 162, which will affect the infusion effect or even cause the infusion device to disable.

In the embodiment of the present invention, a notch 1611 is provided on the end face of the driving wheel 16, close to the reservoir 131, the shape is adapted to the lower movable block 183, and is used for accommodating the lower movable block 183. When lower movable block end 1831 bypasses the arc-shaped stopper 174 and falls into the inside of the arc-shaped stopper 174, the lower movable block 183 is accommodated in the notch 1611 of the driving wheel 161, making full use of the space of the driving wheel body, optimizing the internal design of the infusion mechanism module to reduce the size of the infusion device.

It should be noted here that “inside” and “outside” are relative to the arc-shaped blocking member 174, and belong to the concept of relative position. The positional relationship is as shown in FIG. 9b and FIG. 9d.

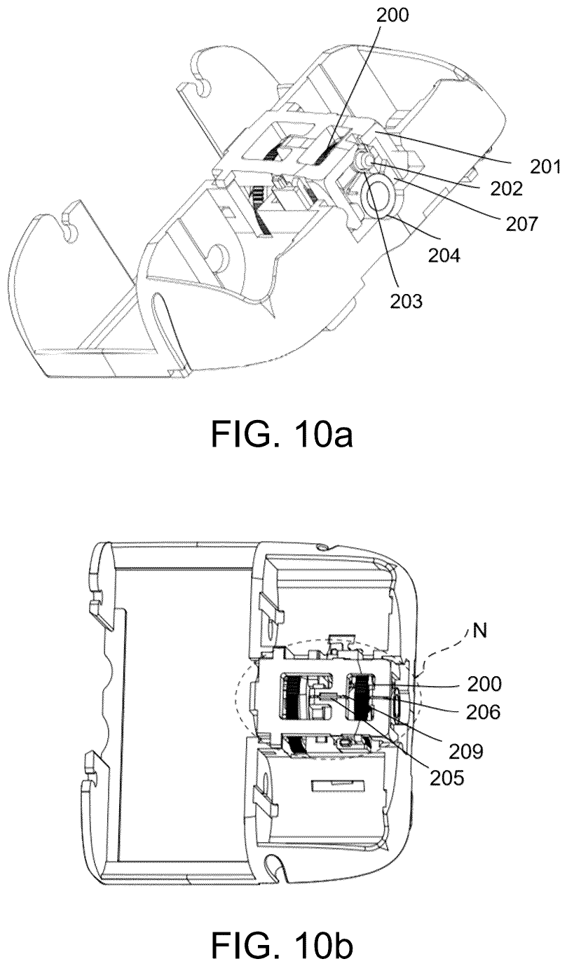

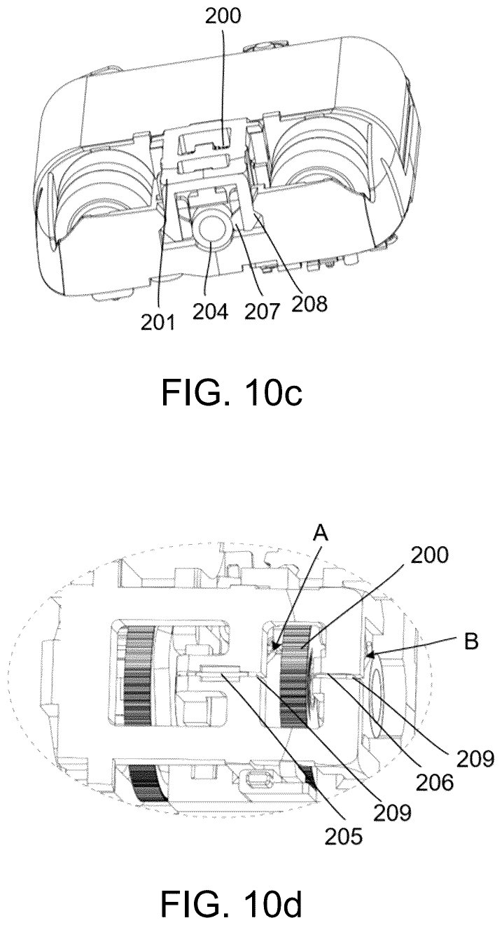

FIG. 10a-FIG. 10c are schematic views of the elastic element and the driving wheel assembly according to the embodiment of the present application. FIG. 10d is a partially enlarged view of the N region of FIG. 10b.

In the infusion device where a single pawl pushes the gear to rotate and completes drug infusion, as shown in FIG. 4c, the pressure exerted by the driving end 1312 on the driving wheel 134 is not uniform at different positions due to the gear teeth and the circumference of the driving wheel 134. Therefore, when the power unit 1311 applies power to the driving end 1312, the driving end 1312 can push the gear teeth to rotate the driving wheel, thereby realizing drug infusion. When the reset unit 1320 applies power to the driving end 1312, the driving end 1312 performs a reset motion, where the drive end 1312 stops pushing the gear, and slides on the surface of the gear teeth. At this time, the drive wheel may rotate in the forward direction or the reverse direction.

In the embodiment of the present invention, the power unit 1311 includes an electrically powered linear driver or an electrically heated linear driver. Specifically, the power unit 1311 is a shape memory alloy. The reset unit 1320 includes an electrically driven linear actuator, an electrically heated linear actuator, or an elastic member. The types of elastic members include, but are not limited to, compression springs, extension springs, torsion springs, elastic sheets, elastic plates, elastic rods, elastic rubbers, and the like. The elastic member can automatically spring back the driving end 1312 to the original position without external force. Specifically, in the embodiment of the present invention, the reset unit 1320 is a torsion spring. The torsion spring is more conducive to the reset of the driving end 1312.

In an embodiment of the present invention, the elastic member is one of a compression spring, a tension spring, and a torsion spring. Preferably, as shown in FIG. 10a, in this embodiment of the present invention, the elastic member is a torsion spring, one end of the driving wheel cover 201 is provided with a cylindrical part 202 for sleeving the torsion spring 203, one end of the torsion spring 203 is abutted against the driving tube 204, and the other end is inserted on the driving wheel cover 201. In other embodiments of the present invention, the other end of the torsion spring 203 can also abut against the driving tube 204, or abut against the side surface (non-gear surface) of the driving wheel 200. By pressing the driving tube 204 or side surface of the driving wheel 200, the maximum static friction force on the driving wheel 200 can be increased, thereby preventing the driving wheel 200 from reversing. The specific abutting position and direction can be adjusted according to the required friction force range. The torsion spring 203 can provide elastic force with larger range, so it can better elastically fix the driving wheel and prevent the driving wheel from reversing.

In another embodiment of the present invention, at least one elastic rod is provided on the driving wheel cover 201. As shown in FIG. 10b and FIG. 10d, the driving wheel cover 201 is provided with a U-shaped elastic rod 205. The “-”-shaped part spans the top of the driving wheel cover 201, one end of the U-shaped elastic rod 205 abuts against the A position of the driving tube 204, and the abutting position on the other side is not shown in the figure. It should be noted that, in the embodiment of the present invention, both ends of the U-shaped elastic rod 205 may fully or partially abut against the side surface of the driving wheel tube 204 or the driving wheel 200. An L-shaped elastic rod 206 is also provided on the driving wheel cover 201. Referring to the directions shown in FIG. 10b and FIG. 10d, the “-”-shaped part of the L-shaped elastic rod 206 straddles the top of the driving wheel cover 201, “The “I” part abuts against the B position of the driving wheel tube 204, and the “I” part may also abut against the side surface of the driving wheel 200. The U-shaped elastic rod 205 and/or the L-shaped elastic rod 206 can increase the maximum static friction force on the driving wheel 200 by pressing the driving tube 204 or the side surface of the driving wheel 200, thereby preventing the driving wheel 200 from reversing. Preferably, a groove 209 is also provided at the top of the driving wheel cover 201, and the U-shaped elastic rod 205 and the L-shaped elastic rod 206 are engaged on the driving wheel cover 201 through the groove 209, so that the U-shaped elastic rod 205 and/or the L-shaped elastic rod 206 can be engaged on the driving wheel cover 201 more stably to provide a more stable elastic friction force range to prevent the driving wheel 200 from reversing, and at the same time, the driving wheel 200 can be elastically fixed. It should be noted that, in the embodiment of the present invention, the specific shape of the elastic rod is not specifically limited, and can be flexibly arranged according to the design features of the driving wheel cover 201, the number of elastic rods, and the abutting position of the elastic rod abutted on the driving wheel assembly is also not particularly limited, and can be adjusted according to the required frictional force range.

In yet another embodiment of the present invention, the elastic member is one or more of silicone, rubber, latex, and spring steel. Preferably, as shown in FIG. 10c, in this embodiment of the present invention, the elastic member is silicone, at least a silica gel 207 is disposed on the cover engaging portion 208 and abuts against the driving tube 204. The silica gel 207 exerts pressure on the driving tube 204 to increase the maximum static friction force received by the driving wheel 200 and prevent the driving wheel 200 from reversing. The silica gel 207 and the cover engaging portion 208 are integrated formed, and are symmetrically abutted on both sides of the drive tube 204 to provide a stable elastic friction force range to prevent the drive wheel 200 from reversing, and at the same time, the driving wheel 200 can be elastically fixed to further reduce the quantity of the components and the size of the infusion device.

It should be noted that, in the above-mentioned embodiments of the invention, for the elastic members, such as the torsion spring 203, the U-shaped elastic rod 205, the L-shaped elastic rod 206 and the silicone 207, arranged on the driving wheel cover 201, the types, quantities, positions, layout such as the abutting position on the driving tube 204 or the driving wheel 200, are not specifically limited here, and can be selected according to actual needs. For example, the torsion spring 203 and the silicone 207 can be selected to provide elastic friction at the same time, as shown in FIG. 10a, as long as the elastic friction force can be provided to prevent the driving wheel 200 from reversing, and the driving wheel 200 can be elastically fixed at the same time.

In yet another embodiment of the present invention, the elastic member and the reset unit 1320 are the same member. Preferably, the reset unit 1320 is a torsion spring, one end is connected to the driving unit 1310, and the other end is abutted against the driving tube 204 or side surface of the driving wheel 200. By applying pressure to the driving tube 204 or the side surface of the driving wheel 200, the maximum static friction force on the driving wheel 200 can be increased, thereby preventing the driving wheel 200 from reversing. The location of the reset unit 1320 on the driving wheel cover 200 is not specifically limited here, and may be provided on the top, side or other positions of the driving wheel cover 200. Therefore, in the embodiment of the present invention, the reset unit 1320 can not only provide elastic force to make the driving end 1312 perform the reset movement, but also can provide elastic friction force to prevent the driving wheel from reversing, and at the same time, it can assist the driving wheel cover 200 to elastically fix the driving wheel 200, further reducing the number of parts and reducing the size of the infusion device.

As a summary, the present invention a drug infusion device, the control mechanism module and the infusion mechanism module are electrically connected to each other by a plurality of first electrical contact and a plurality of second electrical contact that are press againsted each other, optimizing the circuit design and improving the reliability of the electrical connection. In particular, an end portion of the first electrical contact protruds from the surface of the control mechanism module in contact with the first electrical contact, so that when the control mechanism module and the infusion mechanism module are assembled, the first electrical contact and the second electrical contact may be contacted preferentially, further improving the reliability of the electrical connection between the control mechanism module and the infusion mechanism module.

While the invention has been described in detail regarding the specific embodiments of the present invention, it should be understood that it will be appreciated by those skilled in the art that the above embodiments may be modified without departing from the scope and spirit of the invention. The appended claims define the scope of the invention.

1. A drug infusion device, comprising:
a control mechanism, provided with a plurality of first electrical contacts exposed to a surface of the control mechanism; and
an infusion mechanism, comprising a housing, and the infusion mechanism being provided with a plurality of second electrical contacts exposed on a surface of the housing, when the control mechanism and the infusion mechanism are assembled with each other, the first electrical contacts and the second electrical contacts respectively press against each other, thereby electrically connecting the control mechanism with the infusion mechanism; wherein
ends of the first electrical contacts are flush with the surface of the control mechanism or protruding from the surface of the control mechanism in contact with the first electrical contact.

2. The drug infusion device of claim 1, wherein
the control mechanism is provided with a concave, the concave is provided with a recess, and the ends of the first electrical contacts protrude from a surface of the recess.

3. The drug infusion device of claim 1, wherein
the control mechanism is provided with a concave, and the ends of the first electrical contacts protrude from a surface of the concave.

4. The drug infusion device of claim 2, wherein
the first electrical contacts or the second electrical contacts are rigid metal contacts or elastic conductive members.

5. The drug infusion device of claim 4, wherein
the elastic conductive members comprise conductive springs, conductive leaf springs, conductive rubbers or conductive silicone.

6. The drug infusion device of claim 5, wherein
the first electrical contacts are the rigid metal contacts, the second electrical contacts are the conductive springs, ends of the rigid metal contacts are provided with arcuate ends, and when the control mechanism and the infusion mechanism are assembled with each other, the ends of the rigid metal contacts are inserted into inner cavities of the conductive springs.

7. The drug infusion device of claim 3, wherein
the housing is provided with a convex portion for assembly with the concave, and the convex portion is provided with a second groove to avoid interference when the ends of the first electrical contacts protrude from the surface of the concave.

8. The drug infusion device of claim 1, wherein
a buzzer which is non-enclosed is provided in the control mechanism.

9. The drug infusion device of claim 8, wherein
a sound-permeable outlet is provided in the housing of the control mechanism, and a waterproof sound-permeable membrane is disposed between the sound-permeable outlet and the buzzer.

10. The drug infusion device of claim 1, wherein
the control mechanism and the infusion mechanism are detachable, and the control mechanism is reusable.

11. The drug infusion device of claim 1, wherein
the infusion mechanism and the control mechanism are disposed of in one housing, discarded together after a single-use.

12. The drug infusion device of claim 1, further comprising an adhesive patch, wherein the adhesive patch comprises a tape and a protective film, dense ventilation holes are provided on a surface of the tape and arranged evenly.

13. The drug infusion device of claim 1, wherein
the infusion mechanism further comprises a driving unit, a driving wheel and a reservoir, the reservoir is provided with a screw and a piston, a movement of the driving unit drives the driving wheel to rotate, which in turn drives the screw and the piston to move.

14. The drug infusion device of claim 13, wherein
the infusion mechanism further comprises a circuit board or a three-dimensional circuit coated on a part of a surface of a structure, and the circuit board comprises a flexible circuit board or a rigid circuit board.

15. The drug infusion device of claim 14, further comprising a power supply, wherein the power supply is a double-row button battery.

16. The drug infusion device of claim 15, wherein
the infusion mechanism further comprises an elastic conductor, the elastic conductor is electrically connected to the power supply and a specific connection end on the circuit board or three-dimensional circuit.

17. The drug infusion device of claim 16, wherein
the infusion mechanism further comprises a conductive tower-spring, one end of the conductive tower-spring is abutted to the driving unit, an other end of the conductive tower-spring is connected to the specific connection end on the circuit board or three-dimensional circuit.

18. The drug infusion device of claim 3, wherein
the first electrical contacts or the second electrical contacts are rigid metal contacts or elastic conductive members.
